(19) United States
(12) Patent Application Publication
(10) Pub. No.: US 2010/0331929 A1
(43) Pub. Date: Dec. 30, 2010

(54) APPARATUS FOR SPREADING LIGHT FROM MULTIPLE SOURCES TO ELIMINATE VISIBLE BOUNDARIES THEREBETWEEN, LIGHT THERAPY DEVICES INCLUDING SUCH APPARATUS, AND METHODS

(75) Inventors: David Burrows, Orem, UT (US); S. Scott Wheelhouse, Orem, UT (US); Jose Manuel Sasian Alvarado, Tucson, AZ (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/918,631

(22) PCT Filed: Feb. 26, 2009
(86) PCT No.: PCT/IB09/50791

§ 371 Date: Aug. 20, 2010

Related U.S. Application Data
(60) Provisional application No. 61/032,259, filed on Feb. 28, 2008.

(51) Int. Cl. A61N 5/06; G02B 27/30

(52) U.S. Cl. 607/88; 359/641

(57) ABSTRACT

Apparatus for providing substantially uniform illumination with multiple, discrete sources of light include arrays of abutting light spreading elements, such as reflective light pipes or collimation lenses, for substantially eliminating visible boundaries between the sources of light. Methods for generating a substantially uniform field of emission include introducing light into such a spreader array. Light therapy devices including arrays of light spreading elements are also disclosed.

The present invention relates generally to apparatus with arrays of spaced apart light sources. In particular, the present invention relates to light therapy apparatus that include arrays of light sources. More specifically, the present invention relates to light therapy apparatus that include elements for spreading and at least partially collimating light from multiple sources and for providing substantially uniform illumination with multiple sources of light by substantially eliminating visible boundaries between the multiple sources of light.

Over the years, researchers have discovered that light is an effective treatment for a variety of disorders, including jet lag and mild, seasonally-related depression (e.g., seasonal affective disorder, or “SAD”, etc.). A variety of different approaches have been taken in delivering light therapy, such as the use of intense light and the use of light of particular wavelengths (e.g., blue light, green light, etc.).

The apparatus that have been used to deliver light therapy have evolved from large light boxes with fluorescent lights to relatively small, portable devices. Due to their small size and relatively low power consumption requirements, light-emitting diodes (LEDs) are used in many state-of-the-art portable light therapy devices. While arrays of LEDs may be used to provide adequate light therapy, they deliver light in spots

The present invention includes apparatus for providing a substantially uniform field of illumination (e.g., eliminate visible boundaries, reduce visible boundaries to levels where they are not apparent during retinal after-imaging, etc.) with multiple sources of light by substantially eliminating visible boundaries between the multiple sources of light. Such an apparatus may include an array of light spreading elements. The light spreading elements may at least partially collimate light from an array of sources.

One embodiment of such an apparatus includes a reflector array, in which the plurality of light spreading elements are reflectors, or light pipes (e.g., pipe reflectors, solid optically transparent elements that internally reflect light before emitting the light, etc.), that receive and partially collimate light from a corresponding plurality of different, spaced apart light sources. As light is introduced into the reflector, the light is spread in such a way that when viewed from an emission end of the reflector, the light source appears to occupy substantially the entire viewed area of the reflector. Another embodiment of apparatus for providing substantially uniform illumination with light from an array of light sources comprises an array of collimation lenses, with each collimation lens of the array corresponding to a single light source of the array of light sources. Each collimation lens, also referred to herein as a “light spreading element,” effectively magnifies a corresponding light source, spreading the light in such a way that the light source appears to occupy the entire lens.

Various embodiments of methods for spreading light from discrete light sources to generate a substantially uniform field of emission are also within the scope of the present invention.

In another aspect, the present invention includes a light therapy device with an array of light sources and an apparatus for providing substantially uniform illumination from the plurality of light sources.

Other aspects, as well as features and advantages, of the present invention will become apparent to those of ordinary skill in the art through consideration of the ensuing description, the accompanying drawings, and the appended claims.

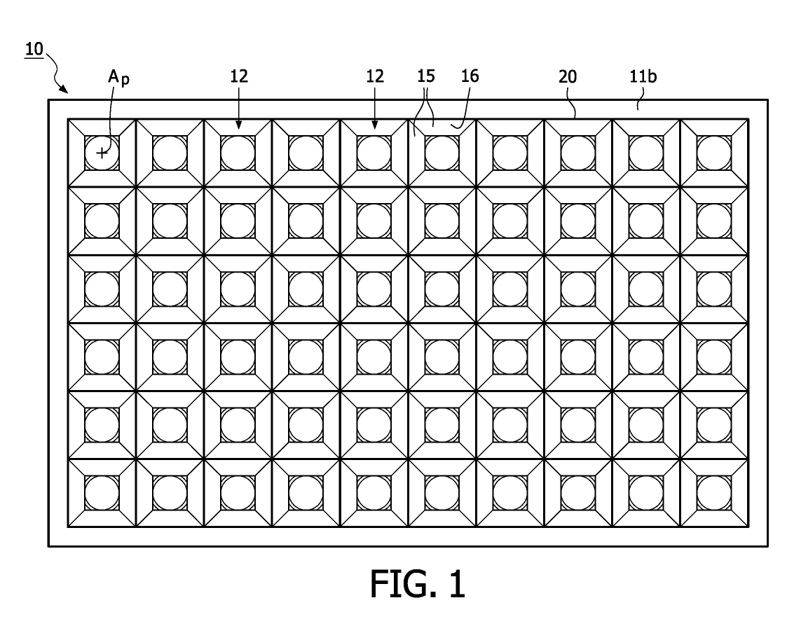

FIG. 1 is a front elevational view of an embodiment of light collimation apparatus according to teachings of the present invention, in which an array of reflectors, or light pipes, collimate light introduced therein and expand the effective, viewed size of a corresponding light source;

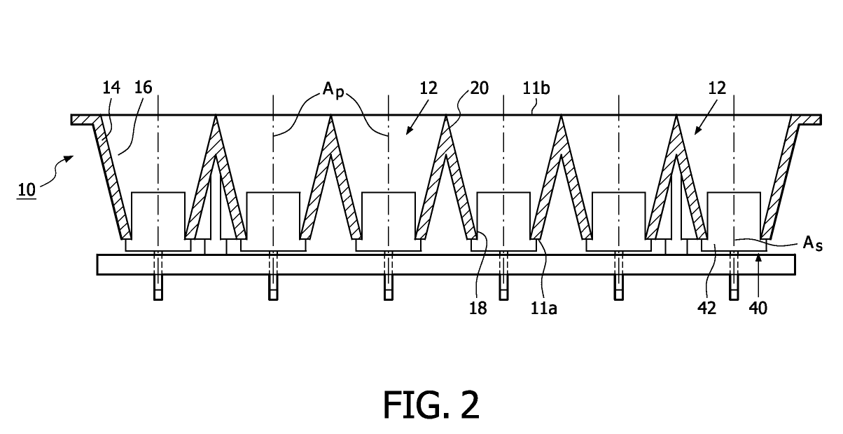

FIG. 2 is a cross-sectional representation of the embodiment of light collimation apparatus shown in FIG. 1, with a light source protruding into each light pipe;

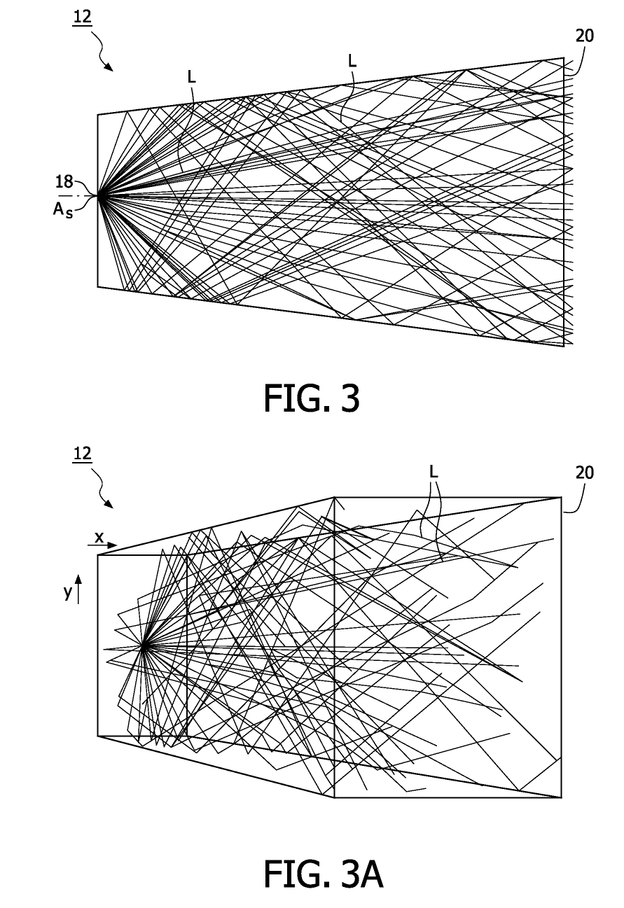

FIGS. 3 and 3A are schematic representations of the affects that a light pipe of an embodiment of a light collimation apparatus of the present invention may have on light introduced therein;

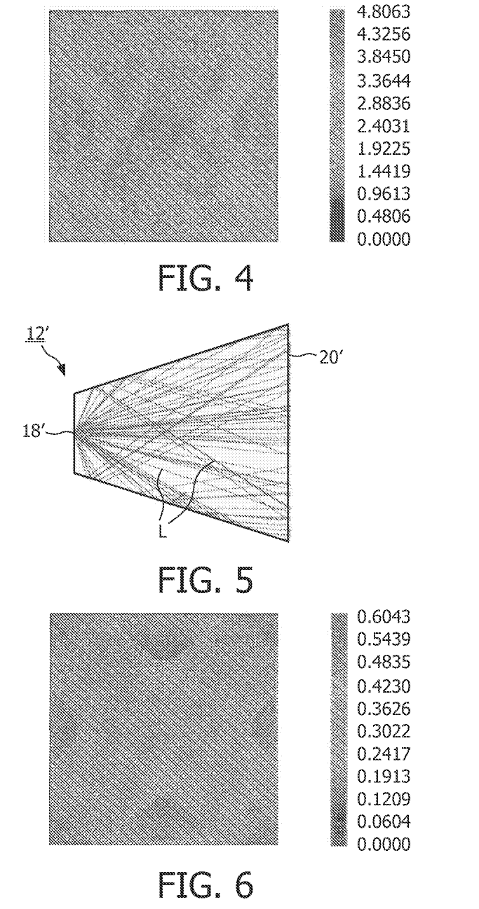

FIG. 4 is an irradiance distribution plot that depicts the spreading of light by a light pipe such as that shown in FIGS. 3 and 3A;

FIG. 5 is a schematic representation of the affect that a light pipe of another embodiment of the present invention has on light introduced therein;

FIG. 6 is an irradiance distribution plot that depicts the spreading of light by a light pipe such as the embodiment shown in FIG. 5;

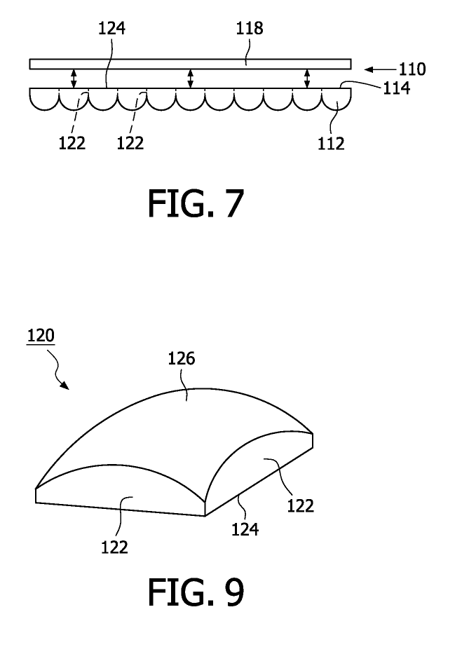

FIG. 7 is a side assembly view of an embodiment of light collimation and diffusion apparatus that incorporates teachings of the present invention;

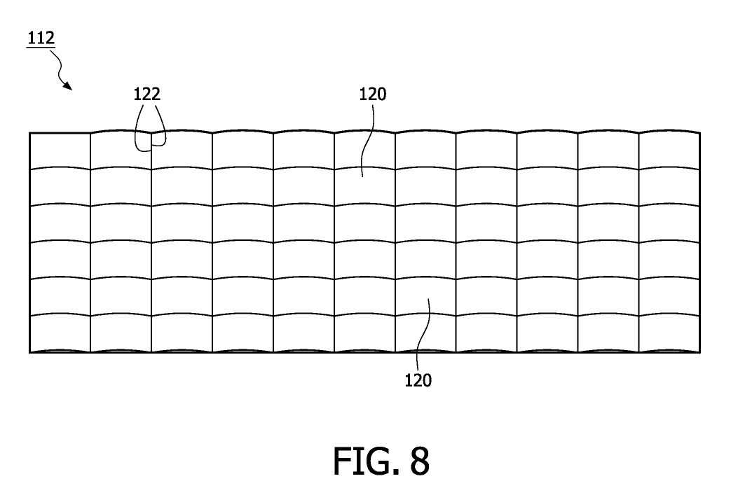

FIG. 8 is a perspective view depicting an embodiment of a collimation element of the apparatus shown in FIG. 7, including an array of collimation lenses;

FIG. 9 is an enlarged perspective view of a collimation lens of the embodiment of collimation element shown in FIG. 8;

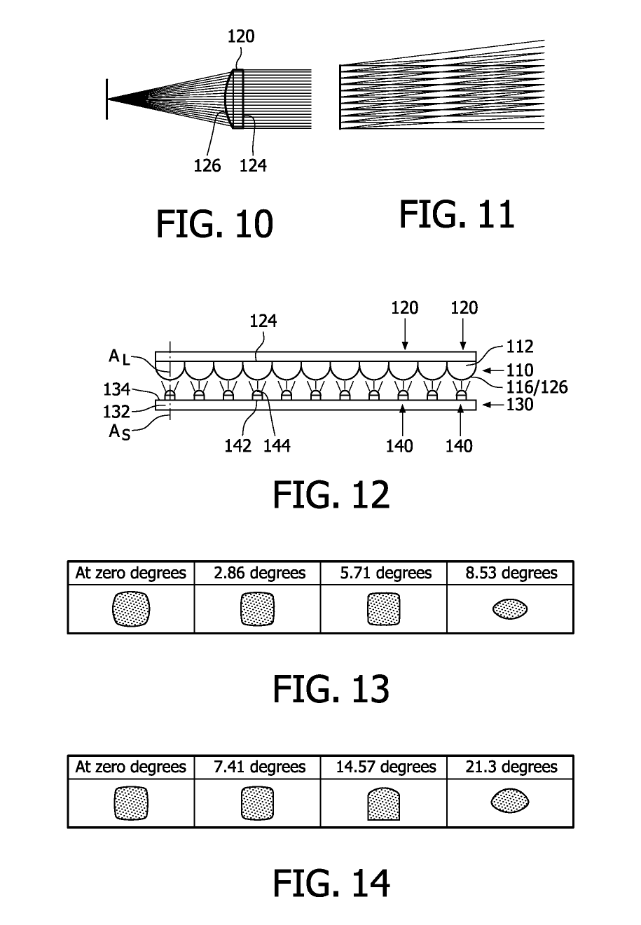

FIG. 10 is a schematic representation of the collimation of light directed through a collimation lens such as that shown in FIG. 9;

FIG. 11 schematically illustrates diffusion of collimated light as the light exits a collimation lens such as that shown in FIG. 9;

FIG. 12 is a side view of a light collimation and diffusion apparatus of the type shown in FIG. 7, which has been assembled with an array of light sources;

FIG. 13 illustrates the degree to which light is spread by a collimation lens such as the embodiment depicted in FIG. 9, as viewed from a variety of angles, when a first embodiment of diffusion element is used in the assembly of FIG. 12;

FIG. 14 illustrates the degree to which light is spread by a collimation lens such as the embodiment depicted in FIG. 9, as viewed from a variety of angles, when a second embodiment of diffusion element is used in the assembly of FIG. 12;

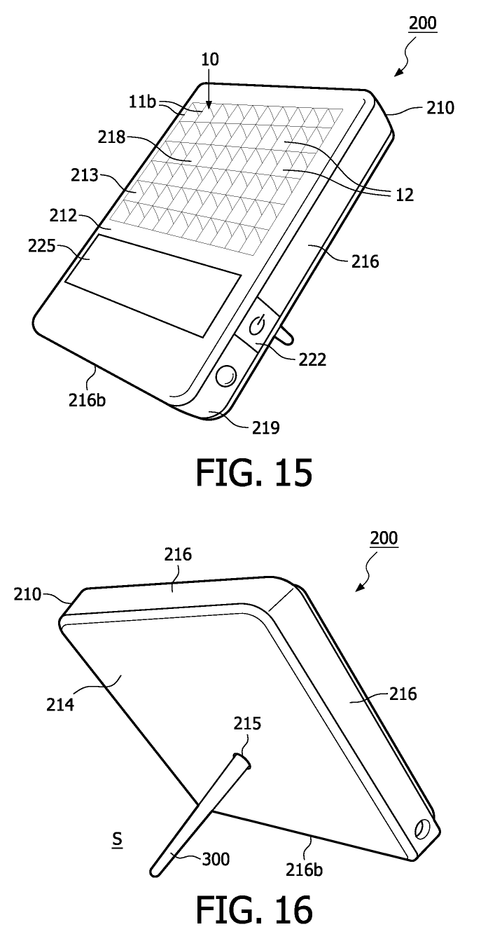

FIG. 15 is a frontal perspective view of an embodiment of a light therapy device that incorporates teachings of the present invention;

FIG. 16 is a rear perspective view of the embodiment of light therapy device shown in FIG. 15;

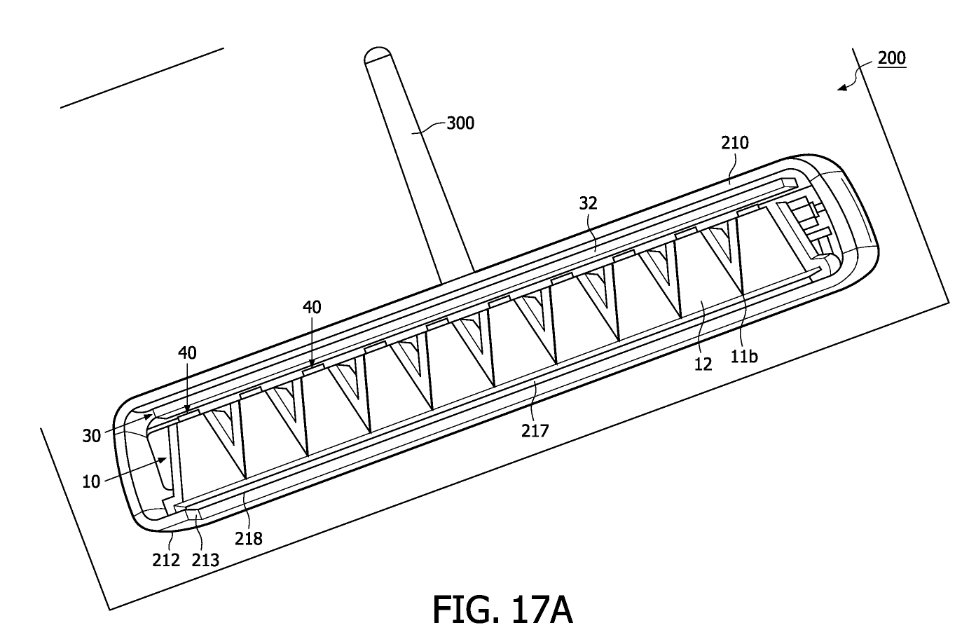

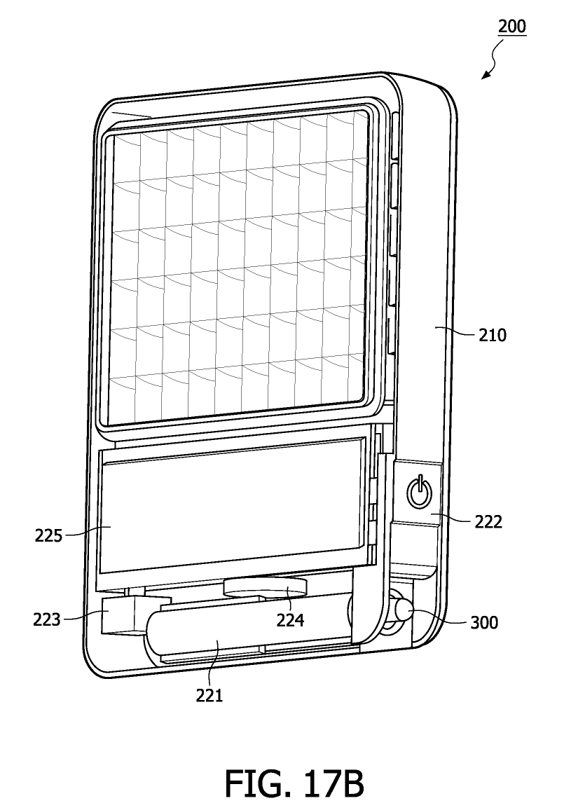

FIGS. 17A and 17B are partial sectional representations of the embodiment of light therapy device of FIGS. 15 and 16, illustrating internal features of the light therapy device;

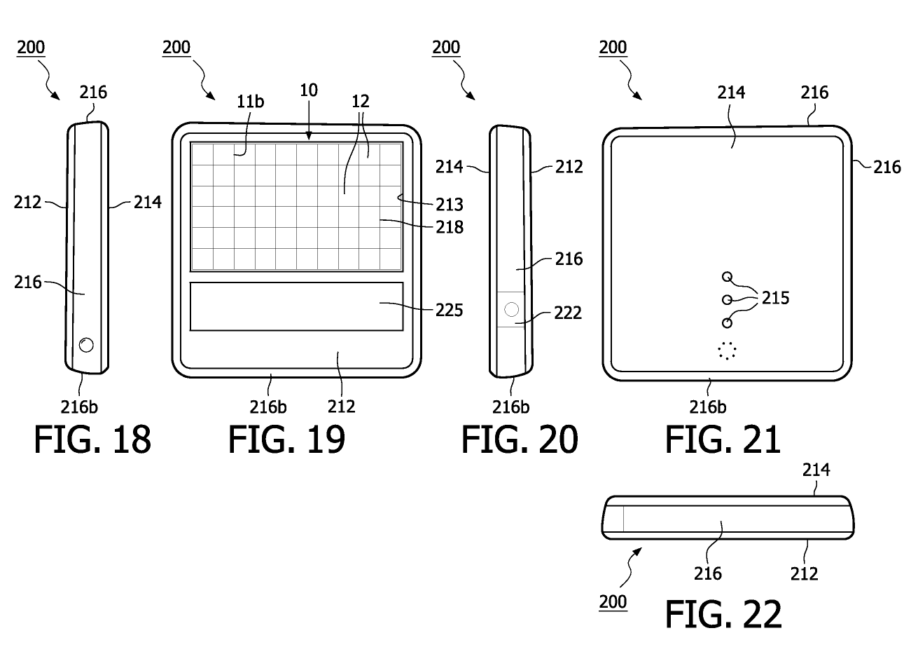

FIGS. 18 through 22 are respectively first side, front, second side, rear, and top plan views of the embodiment of light therapy device shown in FIGS. 15 through 17; and

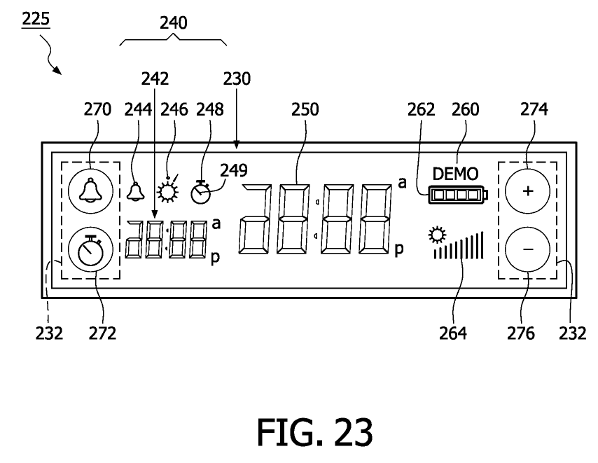

FIG. 23 illustrates an example of a user interface of an embodiment of light therapy device according to the present invention.

An embodiment of a collimation apparatus 10 is shown in FIGS. 1 and 2. Light collimation apparatus 10 includes an array light pipes 12, such as pipe reflectors, solid optical elements that internally reflect light before emitting the light, or other structures that spread light and effectively reduce or eliminate visible boundaries between adjacent light sources. In some embodiments, light collimation apparatus 10 includes a light collection side 11a and an opposite light emission side 11b. Light collection side 11a and light emission side 11b may be oriented substantially parallel to one another, or they may be oriented nonparallel to each other. In other embodiments, one or one or both sides 11a, 11b of light collimation apparatus 10 may be nonplanar (e.g., curved).

Light pipes 12 may be arranged side-to-side, with opposite sides 11a and 11b of light collimation apparatus 10 being formed by opposite ends of the light pipes 12 that form light collimation apparatus 10. Light pipes 12 may be arranged with their axes A_{P} substantially parallel to one another, or they may be arranged with their axes A_{P} oriented at different angles.

Each light pipe 12 includes at least one side wall 14 through which a light passage 16 is defined. In some embodiments, passage 16 may be filled with gas or a mixture of gases (e.g., air). The passages 16 of such embodiments may also be referred to as “open passages.” Other embodiments of light pipe 12 include passages 16 that are filled with optically transparent materials. The passages 16 of these embodiments are also referred to herein as “solid passages.” Light is introduced into passage 16 at one end 18, which is also referred to herein as an “input port,” and exits passage 16 at another, opposite end 20, which is also referred to herein as an “emission port.”

After light is introduced into passage 16 through input port 18. At least some of the light is directed onto the one or more reflective side walls 14 that define passage 16. As that light is reflected by a side wall 14, it is substantially collimated and reflected from the side walls thus smoothing or spreading the light. The degree of collimation depends upon a number of different factors, including, without limitation: the angle or angles, relative to axis A_{P}, which extends along the length of passage 16, at which light is introduced into passage 16; the angle or angles at which each side wall 14 tapers, or is oriented relative to axis A_{P}; the cross-sectional shape or shapes of passage 16, taken along axis A_{P}; the amount of light reflected by each side wall 14 (relative to the amount of light absorbed by that side wall 14); and the surface configuration (e.g., texture) of each side wall 14.

In the illustrated embodiment, each light pipe 12 has a cross section, taken transverse to an axis A_{P} extending through the length of passage 16, that is rectangular in shape. Other cross-sectional shapes are, of course, also within the scope of the present invention. In addition, interior surfaces 15 of each side wall 14 taper slightly outward from input port 18 to emission port 20. The angle at which each side wall 14 tapers relative to a central axis A_{P} of light pipe 12 is less than an angle (e.g., ±80°, ±100°, etc.) at which light is emitted from a light source 40, relative to its central axis A_{S}, that introduces light into light pipe 12. Without limiting the scope of the present invention, some embodiments of light pipes that incorporate teachings of the present invention have side walls that are shaped like a compound parabolic concentrator.

FIGS. 3 and 3A illustrate the substantial collimation of light L that, in reference to FIG. 2, has been emitted by a light source 40 and introduced into passage 16 of such a light pipe 12, with FIG. 3 providing a side view and FIG. 3A providing a perspective view from emission port 20 of light pipe 12. More specifically, FIGS. 3 and 3A depict the partial collimation of light L that has been emitted from an LED 42 having dimensions of 0.125 mm by 0.125 mm with an emission angle, or emission cone, of ±80° in every direction relative to a central axis A_{S} oriented normal to a surface of LED 42, which, in the depicted embodiment, is coincident with axis A_{P} of light pipe 12. LED 42 is positioned at input port 18 of light pipe 12, which, in the depicted embodiment, is 4 mm (e.g., in the x direction shown in FIG. 3A) by 6 mm (e.g., in the y direction shown in FIG. 3A). Light pipe 12 has a length of 16 mm. Upon exiting emission port 20, which has dimensions of 10 mm by 10 mm, light L may be spread only about ±32° (i.e., 4/10×±80°) from axis A_{P} in one (e.g., the x) direction and about ±48° (i.e., 6/10×±80°) from axis A_{P} in the other (e.g., the y) direction.

FIG. 4 is an irradiance distribution plot of a known type, which depicts the intensity of light emitted across the area of emission port 20. As FIG. 4 shows, the light emitted from a 0.125 mm by 0.125 mm LED is diffused over an area of 10 mm by 10 mm (i.e., 100 mm²). The irradiance distribution plot of FIG. 4 is an example of “substantially even” distribution of light and of “substantially uniform” intensity over the area of emission port 20.

For the sake of comparison, FIG. 5 illustrates another embodiment of light pipe 12′. Light pipe 12′ includes a 4 mm by 6 mm input port 18′ and a 16 mm by 16 mm emission port 20′, and has a length of 16 mm. After passing through light pipe 12′, light L from a 0.125 mm by 0.125 mm LED with an emission angle of ±80° in every direction exits light pipe 12′ at an angle of about ±20° in one (e.g., the x) direction (i.e., 4/16×±80°) and about ±30° in the other (e.g., the y) direction (i.e., 6/16×±80°). While the light output from emission port 20′ of light pipe 12′ is more collimated than the light output from emission port 20 of light pipe 12 (FIGS. 3 and 3A), as shown by the irradiance distribution plot of FIG. 6, light pipe 12′ does not diffuse light L (FIGS. 3, 3A, and 5) as uniformly as light pipe 12.

With returned reference to FIGS. 1 and 2, the illustrated embodiment of light collimation apparatus 10 may include a grid array of six light pipes 12 by ten light pipes 12, for a total of sixty light pipes 12. Of course, arrays of different configurations (e.g., hexagonal, etc.) and sizes (i.e., numbers of light pipes 12) are also within the scope of the present invention.

Light collimation apparatus 10 may be made from a variety of materials. In some embodiments, light collimation apparatus 10 may be manufactured from a plastic (e.g., molded, fabricated by photolithographic, stereolithographic, or similar processes, etc.), which may be subsequently coated (e.g., plated, painted, etc.) with a reflective material (e.g., metal, a metallic paint, etc.) In other embodiments, light collimation apparatus 10 may be manufactured (e.g., machined, cast, etc.) from a metal (e.g., aluminum). To further smooth the emitted light, a light diffusion element 118, such as that shown in and described with reference to FIG. 7, may be positioned over light emission side 11b of light collimation apparatus 10 (FIGS. 1 and 2).

Another embodiment of light diffusion and collimation apparatus 110 is shown in FIGS. 7 through 9 and described with reference to FIGS. 7 through 14. As shown in FIG. 7, light diffusion and collimation apparatus 110 includes a collimation element 112 and a diffusion element 118. Collimation element 112, an embodiment of which is depicted in FIG. 8 in an inverted orientation, includes an array (e.g., a grid array, a hexagonal array, etc.) of collimation lenses 120 formed by known processes from any suitable, optically transparent material. In some embodiments, which are referred to herein as “lenticular lens arrays” or, more simply, as “lenticular arrays,” collimation lenses 120 are defined by the coincident high points of two conventional lenticular elements that have been merged (i.e., occupy the same space) with ridges that are oriented perpendicular to one another. Each collimation lens 120 corresponds to a single light source 140 (FIG. 12).

In the embodiment depicted in FIG. 9, in which collimation lenses 120 are arranged in a grid array (see, e.g., FIG. 8), each collimation lens 120 has a substantially planar, rectangular base, or light emission surface 124. In some embodiments, the opposite, light collection surface 126 of each collimation lens 120 may be effectively convex in shape, including shapes that are truly convex, as shown in FIG. 9, as well as and less voluminous, or thinner, flatter Fresnel configurations. The shape of surface 126 may be tailored to provide a desired degree of diffusion and collimation, such as that illustrated by FIG. 10.

In a specific embodiment, each collimation lens 120 has a square light emission surface 124 with sides that are 10 mm long and an aspheric light collection surface 126 with a conic constant of −2.3 and a sag of 4 mm (i.e., the overall thickness of collimation lens 120 is 4 mm). Each collimation lens 120 includes a peripheral boundary 122 extending longitudinally between light emission surface 124 and light collection surface 126. Thus, peripheral boundaries 122 truncate light collection surface 126 short of its full, natural curvature or taper to light emission surface 124. Peripheral boundaries 122 may be provided at locations that cause light from a light source 140 (FIG. 12) that corresponds to collimation lens 120 to visibly occupy an entirety of light emission surface 124. In the depicted embodiment, each collimation lens is a circular, aspheric lens with its edges truncated to provide it with a square periphery. In embodiments where peripheral boundaries 122 of adjacent collimation lenses 120 abut one another (see, e.g., FIGS. 7 and 8), spaces between adjacent light sources 140 may not be visibly apparent when light sources 140 are illuminated.

With returned reference to FIG. 7, diffusion element 118, which may comprise any known type of light diffusion element and may have the form of a thin sheet of plastic material with one or both oppositely facing surfaces thereof textured in a pattern, pseudorandomly, or randomly, is positioned adjacent to light emission surface 124 of each collimation lens 120, or adjacent to light emission surface 114 of collimation element 112, with light emission surface 114 being formed by light collection surfaces 124 of a plurality of adjacent collimation lenses 120. Diffusion element 118 may scatter light that is emitted from collimation element 112 to groups of collimated beams that are oriented at a plurality of different angles within a given range of angles, as shown in FIG. 11. The amount of diffusion depends, of course, at least partially upon the scattering angle of diffusion element 118.

In some embodiments, diffusion element 118 may be separate from collimation element 112. When diffusion element 118 and collimation element 112 are separate from each other, a diffusion element 118 with desired characteristics (e.g., range of diffusion angles and, thus, an area of illumination and brightness, etc.) may be selected from a variety of different diffusion elements 118 for use with collimation element 112 during packaging or use of a light therapy device that includes light collimation and diffusion apparatus 110.

In other embodiments, light emission surface 114/124 may itself be configured to diffuse light and, thus, comprise diffusion element 118.

Turning now to FIG. 12, an example of an assembly that includes an embodiment of light collimation and diffusion apparatus 110 according to the present invention, as well as an array 130 of light sources 140 is illustrated. In the depicted embodiment, array 130 includes a carrier 132, such as a circuit board, with an emission surface 134 that carries light sources 140 and electrically couples them to a power source (not shown) in a manner known in the art. Each light source 140 of the depicted embodiment of array 130 comprises an LED 142 with a lens 144 protruding therefrom. Each light source 140 has a central axis A_{S}, which is oriented normal to a plane of an LED 142 and may extend through a central axis of its lens 144. Lens 144 focuses light emitted by LED 142 such that the light is emitted in the shape of a solid emission cone having a predetermined angle (e.g., 15°, 30°, 45°, etc.) relative to its central axis A_{S}.

Central axes A_{S} of adjacent light sources 140 are spaced apart from one another by a distance that corresponds to the distance that adjacent axes A_{L} of corresponding collimation lenses 120 are spaced apart from each other. In addition, light sources 140 are arranged in a manner (e.g., in a grid array) that corresponds to the arrangement their corresponding collimation lenses 120 of collimation element 112. Accordingly, central axes A_{S} of all of the light sources 140 of array 130 and central axes A_{L} of all of their corresponding collimation lenses 120 may be aligned and coincident with each other.

An emission surface 132 of array 130 faces a collection surface 116 of collimation element 110. The distance between a surface of an LED 142 of array 130 and the focal point of its corresponding collimation lens 120 of collimation element 112 may be tailored in such a way as to optimize or maximize the collection of light by collection surface 126 of collimation lens 120, as well as optimize or maximize the diffusion of light by emission surface 124 of collimation lens 120.

FIGS. 13 and 14 depict examples of the amount of diffusion that may be achieved with different embodiments of light collimation and diffusion apparatus 110. In both of these examples, collimation element 112 (FIGS. 7 and 8) includes collimation lens 120 with square light emission surfaces 124 having sides that are 10 mm long and an aspheric light collection surface 126 with a conic constant of −2.3 and a sag of 4 mm. Collimation element 112 is, as shown in FIG. 12, assembled with an array 130 of light sources 140 with LEDs that are 5 mm across and lenses 144 that focus light to an angle, or emission cone, of within about 15° of a central axis A_{S} of each light source 140. A surface of an LED 142 of each light source 140 is positioned 12 mm from a focal point of the collimation lens 120 that corresponds to that light source 140, providing for a focal length of 12 mm and an F/number (F/#) of 1.2, which is equal to the focal length divided by the distance across (e.g., diameter, etc.) collimation lens 120.

FIG. 13 depicts the spreading of light when such a collimation element 112 (FIG. 12) is used with a diffusion element 118 (FIG. 12) having a scattering angle of 10°, as perceived at a distance of 500 mm. When emitted light is viewed from a location directly above (i.e., inline with a central axis A_{L} through) a collimation lens 120 (FIGS. 8 and 9), the light spreads substantially uniformly and entirely to the peripheral boundaries 122 (FIGS. 8 and 9) of emission surface 124 of collimation lens 120, with only small areas at the corners of collimation lens 120 not being fully illuminated. As the lateral distance of the location over a collimation lens 120 from which light is viewed increases, the perceived amount of light spreading by that collimation lens 120 decreases, gradually at first, then more dramatically. In this regard, FIG. 13 also illustrates the perceived spreading of certain lateral offset distances and their corresponding angles relative to a central axis A of collimation lens 120, with 2.86° corresponding to a lateral offset distance of 25 mm from axis A, 5.71° corresponding to a lateral offset distance of 50 mm from axis A, and 8.53° corresponding to a lateral offset distance of 75 mm from axis A. At lateral offset distances of 25 mm and 50 mm, light is still spread substantially across the area of emission surface 122 of each collimation lens 120.

The perceived amounts of light spreading that are shown in FIG. 14 occur when the above-described embodiment of collimation element 114 is used with a diffusion element 118 having a scattering angle of 25°. At viewing angles of 0° (no lateral offset at a longitudinal distance of 500 mm from emission surface 122 (FIG. 9)), 7.41° (lateral offset of 65 mm at a longitudinal distance of 500 mm), and 14.57° (lateral offset of 130 mm at a longitudinal distance of 500 mm), the light appears to be spread substantially uniformly across the area of emission surface 122. By the point where the viewing angle increases to 21.3° (lateral offset of 195 mm at a longitudinal distance of 500 mm) however, the light no longer appears to have been spread across the entire area of emission surface 122.

Of course, other arrangements are also within the scope of the present invention, including arrangements including LEDs that emit light at smaller or larger angles (i.e., in emission cones with different angles relative to their axes), as well as arrangements with different focal lengths.

Variations of the disclosed embodiments of light collimation apparatus (e.g., “folded” light pipes in which light is initially reflected by a partially reflective, partially transmissive material back toward the light sources and onto reflective surfaces of side walls of the light pipe; superimposed lenticular films with elongate lenses oriented nonparallel (e.g., perpendicular, etc.) to each other, etc.) are also within the scope of the present invention, as are other embodiments of apparatus and systems for collimating and spreading light to substantially eliminate visible borders between adjacent light sources (e.g., other optically transparent elements with features, such as semicylindrical lenses, semispherical lenses, embedded beads, or surface features that effectively enlarge the appearance of a plurality of adjacent light sources and substantially eliminate visible boundaries between the light sources; etc.).

In use, various embodiments of light collimation and diffusion apparatus of the present invention may be included in light therapy devices. An example of such a light therapy device 200 is shown in FIGS. 15 through 23.

For the sake of simplicity, light therapy device 200 is illustrated by FIGS. 15 through 17 and 19 as including particular embodiments of light collimation and diffusion apparatus 10 and light sources 40. It should be recognized, however, that any other suitable embodiments of these elements, including, but not limited to, light collimation and diffusion apparatus 110 and array 130 of light sources 140 (FIGS. 7 through 10 and 12), may be substituted for the illustrated embodiments.

With reference to FIGS. 15 through 22, light therapy device 200 includes a housing 210 with a front side 212, an opposite back side 214, and peripheral edges 216. At least one peripheral edge 216b serves as a base upon which housing 210 may rest when supported upon a surface, such as a table top or desk top.

Housing 210 may, in some embodiments, have dimensions that make it portable. In a particular embodiment, a thickness of housing 210 (i.e., the distance between front side 212 and back side 214) may be about one inch or less (e.g., about 0.950 inch, or about 2.5 cm) a height of about 5.25 inches (about 13.3 cm) and a width of about 5.4 (about 13.7 cm) inches.

With specific reference to FIGS. 15 and 19, a light emission window 213 is located in front side 212 of housing 210. An optically transparent element 218 may be disposed in light emission window 213. Optically transparent element 218 may, in some embodiments, comprise a light diffuser, a lens, or simply a planar element.

With continued reference to FIGS. 15 and 19, and added reference to FIG. 17A, housing 210 contains a light collimation and diffusion apparatus 10 and an array 30 of light sources 40. Light collimation and diffusion apparatus 10 and array 30 are assembled in such a way that light sources 40 introduce light into elements (e.g., light pipes 12, etc.) of light collimation and diffusion apparatus 10. A light emission side 11b of light collimation and diffusion apparatus 10 is oriented toward and may be visible through window 213. Optically transparent element 218, if present, extends across window 213 to protect the elements of light therapy device 200 (e.g., light collimation and diffusion apparatus 10, array 30, etc.) that are contained within housing 200. In embodiments where light emission side 11b of light collimation and diffusion apparatus 10 is spaced apart from transparent element 218, such as that shown in FIG. 17A, air or other gases within that space 217 may further diffuse light exiting emission side 11b. As noted above, in some embodiments, optically transparent element 218 may provide for additional diffusion of light that exits light emission side 11b of light collimation and diffusion apparatus 10.

In addition to containing a light collimation and diffusion apparatus and an array of light sources, as shown in FIG. 17B, housing 210 may contain and/or carries a number of other features of light therapy device, such as a storage receptacle 221 for stand 300. Housing 210 may also contain a variety of electronic elements, including, without limitation, one or more user interface elements 225 (e.g., a touch-sensitive display or other input and output elements) (see also FIG. 23) a control circuit board (not shown, but may be located behind user interface element 225) that carries one or more of a processor, other control elements (e.g., resistors, capacitors, diodes, transistors, inductors, etc.), and a switch, a battery in communication with electronic features on the circuit board and with user interface element 225 (not shown, but may be located behind one or both of storage receptacle 221 and user interface element 225), and a power supply plug 223 accessible from an exterior of housing 210 and electrically coupled to the battery. A variety of other features, such as the illustrated speaker 224, cooling elements, and the like, may also be contained within or otherwise carried by housing 210. One or more of the elements within housing 210 may be accessed by way of a door 219 (FIG. 15).

FIG. 23 illustrates a nonlimiting example of a user interface element 225 that may be included in an embodiment of a light therapy device 200 (FIGS. 15 through 22) of the present invention. In the depicted example, user interface element 225 is a touch-sensitive liquid crystal device that includes a non-sensitive display region 230 and touch-sensitive control region 232, both of which communicate with and may be controlled by programming of a processor (not shown) of light therapy device 200 in any suitable, known manner.

Display 230 of user interface element 225 may include one or more features. In the embodiment shown in FIG. 23, display 230 includes timer features 240, a digital clock 250, and indicator features 260.

Timer features 240 of display 230 include a digital display 242 and icons 244, 246, 248. Depending upon a display mode that has been selected by a user, digital time display 242 may show a time at which an alarm is to sound, a time at which light sources 40 (FIG. 17A) are to be automatically illuminated, a duration of time that has been set for illumination of light sources 40, or an amount of illumination time remaining.

Icons 244, 246, 248 may provide information to a user about whether or not certain functionalities of light therapy device 200 (FIGS. 17A through 22) are active, or have been turned “on” or “off.” As an example, an icon 244, 246, 248 may appear, or be visible, on display 230 if a particular, corresponding functionality his active, or turned “on”. If that functionality is inactive, or has been turned “off”, the icons 244, 246, 248 may not appear on display, or may appear distinctively lighter than icons 244, 246, 248 that correspond to functionalities that are active.

Optionally, icons 244, 246, 248 may be configured to provide an indication of an active programming and/or display mode. In some embodiments, icons 244, 246, 248 may pulse, or “blink,” or display 230 may otherwise signify (e.g., with a visible graphic element located around or next to an icons 244, 246, 248) a particular functionality of light therapy apparatus 200 that corresponds to a value shown on digital display 242.

In the illustrated embodiment, icon 244 appears as a bell when an audible alarm function (i.e., an alarm clock) of light therapy device 200 has been activated, or is turned “on.” Inidicium 244 may also signify when digital display 242 shows the time that an audible alarm is to sound.

Icon 246, which graphically depicts a sun in the illustrated embodiment, provides an indication of whether or not a visible alarm function (e.g., illumination of one or more light sources 40 (FIG. 17A) is active, or “on.” Additionally, icon 246 may provide an indication of instances where the time shown by digital display 246 is the time at which light therapy apparatus 200 is programmed to illuminate light sources 40.

Icon 248, which has the appearance of a stopwatch in the illustrated embodiment, appears when light sources 40 have been activated and will remain activated for a predetermined (e.g., under control of a timer) period of time. In embodiments where icon 248 appears as a stopwatch, icon 248 may include a hand 249 or another feature, such as a digital value associated with the graphically depicted stopwatch, that may provide an indication of the time (e.g., in minutes) remaining before the timer counts down to zero and illumination of light sources 40 is terminated. In addition, icon 248 may be configured to provide an indication of instances in which digital display 242 provides a count-down of a duration of time that light sources 40 will remain illuminated.

Other embodiments of icons 244, 246, 248, as well as icons that correspond to different functions are, of course, also within the scope of the present invention.

Indicator features 260, 262, 264 may be respectively configured to indicate a variety of features, such as a current mode (e.g., “DEMO”, programming, manual, automatic, dawn simulation, etc.) of light therapy device 200 (FIGS. 15 through 22), an amount of battery power remaining, and an intensity of light emitted relative to the potential intensity of light that may be emitted by light sources 40.

Touch-sensitive control regions 232 of user interface element 225 may include one or more “buttons” 270, 272, 274, 276 that enable an individual to operate light therapy device 200 (FIGS. 15 through 22). In the illustrated embodiment, buttons 270 and 272 allow an individual to select a mode displayed by display region 230, while buttons 274 and 276 enable the user to select a degree of illumination and to otherwise program a processor (not shown) that controls the operation of light therapy device 200.

With reference now to FIGS. 16 and 21, an example of the manner in which a light therapy device 200 according to the present invention may be supported is depicted. In the depicted example, a base 216b of housing 210, together with a stand 300 (which may, in a specific embodiment, have a length of about 2.8 inches (about 7.1 cm)) that may be removably inserted into one or more apertures 215 in back side 214 of housing may support light therapy device 200 upon a surface S. Stand 300 may comprise an elongate element, such as a pin or peg, with a length that, when inserted in an aperture 215, orients a front side 212 of light therapy apparatus 200 at a nonperpendicular angle relative to surface S (see FIG. 15). In embodiments where multiple apertures 215 are formed at different longitudinal locations in back side 214, a single stand 300 may be used to orient front side 214 at a variety of different angles, depending upon the particular aperture 215 with which stand 300 is assembled.

Other features for supporting housing 210 of light therapy apparatus 200 and for orienting the same at a desired angle relative to a surface S (FIG. 16), such as pivotal legs, retractable features, and the like, of course, may also be used with a light therapy device that incorporates teachings of the present invention.

Although the foregoing description contains many specifics, these should not be construed as limiting the scope of the present invention, but merely as providing illustrations of some embodiments. Similarly, other embodiments of the invention may be devised which do not exceed the scope of the present invention. Features from different embodiments may be employed in combination. The scope of the invention is, therefore, indicated and limited only by the appended claims and their legal equivalents, rather than by the foregoing description. All additions, deletions and modifications to the invention as disclosed herein which fall within the meaning and scope of the claims are to be embraced thereby.

1. A light collimation apparatus for use with an array of light sources, comprising an array including a plurality of light pipes oriented in side-by-side relation to each other, each light pipe of the plurality including a passage defined by at least one side wall with an at least partially reflective surface.

2. The light collimation apparatus of claim 1, wherein the plurality of light pipes are arranged in a grid array.

3. The light collimation apparatus of claim 1, wherein the plurality of light pipes are oriented substantially parallel to one another.

4. The light collimation apparatus of claim 1, wherein each light pipe includes an input port at one end and an emission port at an opposite end.

5. The light collimation apparatus of claim 4, wherein the at least one side wall of each light pipe tapers outwardly from the input port to the emission port.

6. The light collimation apparatus of claim 5, wherein each input port has a rectangular cross section.

7. The light collimation apparatus of claim 5, comprising a compound parabolic concentrator.

8. The light collimation apparatus of claim 1, wherein the passage comprises an open passage.

9. The light collimation apparatus of claim 1, wherein the passage comprises a solid passage.

10. A light therapy apparatus, comprising:
an array of discrete, spaced apart light sources; and
a light collimation apparatus, including an array of light pipes, each light pipe of the array positioned to receive light from a corresponding light source of the array of light sources.

11. The light therapy apparatus of claim 10, wherein each light pipe of the array includes a side wall defining a passage therethrough.

12. The light therapy apparatus of claim 11, wherein the passage comprises an open passage.

13. The light therapy apparatus of claim 11, wherein the passage comprises a solid passage.

14. The light therapy apparatus of claim 10, wherein the array of discrete, spaced apart light sources comprises an array of light emitting diodes.

15. The light therapy apparatus of claim 14, wherein each light emitting diode of the array has an emission angle that exceeds an angle at which a side wall of its corresponding light pipe tapers.

16. The light therapy apparatus of claim 15, wherein each light emitting diode is positioned at the input port of its corresponding light pipe.

17. The light therapy apparatus of claim 10, further comprising:
a diffusion element positioned over a light emission side of the light collimation apparatus.

18. The light therapy apparatus of claim 18, wherein each light pipe spreads light from a single light emitting diode substantially evenly over an area larger than an area of the light emitting diode.

19. The light therapy apparatus of claim 18, wherein at least some light pipes of the array are configured and oriented to substantially remove visible borders between light emitted from adjacent light sources of the array.

20. The light therapy apparatus of claim 10, further comprising:
a housing, a front side of the housing including a light emission window through which light exiting the light diffusion and collimation apparatus is directed.

21. The light therapy apparatus of claim 20, wherein the housing has a thickness of about one inch or less.

22. The light therapy apparatus of claim 20, wherein at least one support receptacle is defined in a back side of the housing.

23. The light therapy apparatus of claim 22, further comprising:
a support element configured to be received by the at least one support receptacle.

24. The light therapy apparatus of claim 23, wherein the support element is configured to orient the light emission window at an angle relative to a surface by which the housing and the support element rest.

25. The light therapy apparatus of claim 24, wherein the housing includes a plurality of support receptacles positioned a plurality of distances from a base of the housing for orienting the light emission window at a plurality of different angles relative to the surface.

26. The light therapy apparatus of claim 23, wherein the support element is enlongate.

27. The light therapy apparatus of claim 26, wherein the support element extends substantially linearly.

28. An apparatus for spreading light from an array of discrete light sources, comprising:
an array of light spreading elements, each light spreading element aligned with a light source of the array of discrete light sources and configured to spread light introduced therein to substantially an entire periphery of a light emission end of the light spreading element so the light source appears to occupy an entirety of the light emission end and visible boundaries between the discrete light sources are substantially eliminated.

29. The apparatus of claim 28, wherein the array of light spreading elements comprises a grid array with each light spreading element having a rectangular light emission end.

30. The apparatus of claim 28, wherein the array of light sources comprises an array of collimation lenses with truncated edges that abut one another.

31. The apparatus of claim 28, wherein the array of light sources comprises an array of optically reflective light pipes.

32. A light therapy apparatus, comprising:
a light array including a plurality of discrete light sources; and
a spreader array including a plurality of light spreading elements, each light spreading element aligned with a light source of the light array and configured to spread light introduced therein to substantially an entire periphery of a light emission end of the light spreading element so the light source appears to occupy an entirety of the light emission end and an emission surface of the spreader array is substantially uniformly illuminated.

33. The light therapy apparatus of claim 32, further comprising:
a diffusion element positioned over light emission ends of each light spreading element of the array of light spreading elements.

34. The light therapy apparatus of claim 32, further comprising:
a housing carrying the light array and the spreader array and including a window through which the light emission surface of the spreader array is visible.

35. The light therapy apparatus of claim 34, further comprising:
at least one elongate support element, wherein at least one aperture is recessed within a the housing to receive the at least one elongate support.

36. The light therapy apparatus of claim 35, wherein a plurality of apertures are recessed within the housing at different longitudinal locations to enable selection of a plurality of housing orientation angles with the same elongate support.

37. The light therapy apparatus of claim 35, further comprising a storage receptacle for receiving the elongate support.

38. A method for providing light therapy, comprising:
illuminating a plurality of discrete light sources;
directing light from the plurality of discrete light sources into a corresponding plurality of light spreading elements, within which the light is at least partially collimated and spread; and
emitting light substantially uniformly across an area of an emission end of each light spreading element of the plurality.

39. The method of claim 38, wherein directing light comprises directing light into an array of light spreading elements with peripheral edges that abut one another and wherein emitting light includes emitting light substantially uniformly across an emission surface of the array.
